(19) United States
(12) Patent Application Publication
(10) Pub. No.: US 2018/0280605 A1
(43) Pub. Date: Oct. 4, 2018

(54) ARTERIOVENOUS GRAFT FOR MINIMIZING ARTERIAL STEAL AND GRAFT THROMBOSIS

(71) Applicant: InnAVasc Medical, Inc., Durham, NC (US)

(72) Inventors: Shawn M. Gage, Raleigh, NC (US); Jeffrey H. Lawson, Durham, NC (US); Joseph Knight, Durham, NC (US); Craig Nichols, Carrboro, NC (US)

(21) Appl. No.: 15/941,790

(22) Filed: Mar. 30, 2018

Related U.S. Application Data
(60) Provisional application No. 62/479,782, filed on Mar. 31, 2017.

(51) Int. Cl. A61M 1/36; A61M 39/02

(52) U.S. Cl. CPC A61M 1/3655; A61M 39/0247; A61M 2039/0297; A61M 2039/027; A61M 2039/0276; A61M 2039/0258

(57) ABSTRACT

An arteriovenous dialysis access graft is configured to be implanted in a body of a subject. The arteriovenous dialysis graft comprises a flexible conduit defining a longitudinal flow passageway. The conduit has a first end portion and a second end portion, the first end portion configured to connect to an artery of the subject and the second end portion configured to connect to a vein of the subject such that blood flows through the longitudinal flow passageway of the conduit from the first end portion to the second end portion. The graft further comprises a first cannulation chamber and a spaced second cannulation chamber with the conduit extending through each of the first chamber and the second chamber. The first chamber is positioned between the first end portion and the second chamber, and the second chamber is positioned between the second end portion and the first chamber. A valve device is positioned between the first cannulation chamber and the second cannulation chamber for controlling fluid flow.

### CROSS-REFERENCES

This application is related to U.S. provisional application No. 62/479,782, filed Mar. 31, 2017, entitled “ARTERIOVENOUS GRAFT FOR MINIMIZING ARTERIAL STEAL AND GRAFT THROMBOSIS”, naming Shawn M. Gage and Jeffrey H. Lawson as the inventors. The contents of the provisional application are incorporated herein by reference in their entirety, and the benefit of the filing date of the provisional application is hereby claimed for all purposes that are legally served by such claim for the benefit of the filing date.

### BACKGROUND

An arteriovenous graft is described for minimizing arterial steal and graft thrombosis and, more particularly, an arteriovenous graft having means for controlling blood flow through the graft is provided.

Vascular access for hemodialysis is provided by subcutaneous placement of an arteriovenous graft. The arteriovenous graft comprises a tubular element made from biocompatible material, for instance, a fluoropolymer such as polytetrafluoroethylene. One end of the arteriovenous graft is connected to an artery while the other end is connected to a vein. Blood flows from the artery through the graft and into the vein. To connect the patient to a dialysis machine, two large hypodermic needles are inserted through the skin and into the graft. Blood is removed from the arteriovenous graft through one needle, circulated through the dialysis machine, and returned to the patient through the second needle. Typically, patients undergo hemodialysis approximately four hours per day, three days per week.

Problems have been experienced with the use of an arteriovenous graft. For example, “arterial steal” occurs when excessive blood flow through the arteriovenous graft “steals” blood from the distal arterial bed. Arterial steal can prevent the proper supply of blood from reaching the extremity of a patient. In addition, blood flowing through the arteriovenous graft can often reach turbulent flow rates, which then exits the arteriovenous graft and contacts the vein connected to the graft. This collision between the blood and the vein may cause the development of neointimal hyperplasia, which leads to the thickening of the vein walls and a narrowing of the vessel. As the vein narrows, flow through the arteriovenous graft decreases and blood within the graft may ultimately clot. The cessation of blood flow through the graft caused by clot formation is known as graft thrombosis.

For the foregoing reasons, there is a need for an arteriovenous graft that can minimize arterial steal and graft thrombosis. Ideally, the new arteriovenous graft will prevent high flows through the graft while it is not being used and thus reducing or eliminating the stimulus for intimal hyperplasia.

### SUMMARY

An arteriovenous dialysis access graft is provided to be implanted in a body of a subject. The arteriovenous dialysis graft comprises a flexible conduit defining a longitudinal flow passageway, the conduit having a first end portion and a second end portion. The first end portion is configured to connect to an artery of the subject and the second end portion is configured to connect to a vein of the subject such that blood flows through the longitudinal flow passageway of the conduit from the first end portion to the second end portion. A first cannulation chamber and a spaced second cannulation chamber are provided. The conduit extends through each of the first chamber and the second chamber. The first chamber is positioned between the first end portion and the second chamber, and the second chamber is positioned between the second end portion and the first chamber. A valve device is positioned between the first cannulation chamber and the second cannulation chamber for controlling fluid flow.

In one aspect, the valve device comprises an inner sleeve positioned within an outer sleeve, and an actuator in flow communication with the fluid passageway defined through the outer sleeve of the valve device. The actuator is configured to open and close the valve device when fluid is directed through the fluid passageway and against the wall portion such that the wall portion inflates so as to form a balloon that restricts the flow of blood through the arteriovenous graft. The actuator may be positioned outside of the arteriovenous graft and comprise a fluid injection port that is in fluid communication with the valve device.

In a further aspect, the outer sleeve of the valve device is more rigid than the inner sleeve and the outer sleeve maintains its shape when the respective balloon is inflated.

In one embodiment, the actuator comprises a piston that pumps the fluid to the valve device. In this aspect, the actuator comprises a fluid delivery device that delivers the fluid to the valve device for opening and closing the valve device. The fluid comprises a liquid or a gas.

In another aspect, each chamber comprises an elongated chamber body surrounding the conduit. The chamber body comprises an annular inner layer including self-sealing material surrounding the conduit. An outer layer is around the inner layer and defines a cannulation port that exposes the self-sealing material. An elongated shell is embedded in the chamber body between the inner layer of the chamber body and the outer layer of the chamber body and extends generally parallel to the longitudinal flow passageway of the conduit. The shell includes a posterior wall and a pair of sidewalls defining an open anterior portion facing the cannulation port of the chamber body. Each shell is formed of a substantially rigid material such that, when a dialysis needle is inserted through the cannulation port and the self-sealing material, the needle is inhibited or prevented from extending through the posterior or the side walls of the shell.

### BRIEF DESCRIPTION OF THE DRAWINGS

For a more complete understanding of the arteriovenous graft, reference should now be had to the embodiments shown in the accompanying drawings and described below. In the drawings:

FIG. 1 is a perspective view of an embodiment of an arteriovenous graft including a valve device and a flow sensor along a body portion of the arteriovenous graft.

FIG. 2 is a transverse cross-section view of a portion of the arteriovenous graft taken along line 2-2 of FIG. 1 with a valve shown in an open position and the valve device removed for clarity.

FIG. 3 is a transverse cross-section view of a portion of the arteriovenous graft as shown in FIG. 2 with the valve shown in the closed position.

FIG. 4 is a perspective view of another embodiment of an arteriovenous graft including a valve device between a pair of cannulation chambers.

FIG. 5 is a schematic view of the arteriovenous graft as shown in FIG. 4 with the valve shown in a closed position.

FIG. 6 is a schematic view of the arteriovenous graft as shown in FIG. 4 with a valve device at an arterial end of the arteriovenous graft with the valve shown in a closed position.

FIG. 7 is a schematic view of the arteriovenous graft as shown in FIG. 6 including a narrow diameter body portion at an arterial end of the arteriovenous graft adjacent the valve device with the valve shown in a closed position.

FIG. 8 is a longitudinal cross-section view of a third embodiment of an arteriovenous graft including a pair of valve devices spaced along a body portion of the arteriovenous graft.

FIG. 9 is an up-close longitudinal cross-section view of a portion of an arteriovenous graft including a variable diameter lining along a length of the arteriovenous graft.

FIGS. 10A and 11B are transverse cross-sectional views of the arteriovenous graft including the valve device as shown in FIG. 1 wherein the valve device is in an open position and a closed position, respectively.

FIGS. 11A and 11B are up-close longitudinal cross-section views of a cannulation chamber of the arteriovenous graft as shown in FIG. 4 including a valve device providing a variable diameter inner surface wherein the valve device is in an open position and a closed position, respectively.

FIGS. 12 and 13 are schematic views of actuators including a piston for actuating a valve device for use in an arteriovenous graft

FIG. 14 is a schematic view of a portion of an arteriovenous graft including a valve device comprising an external injection port.

FIG. 15 is a flow diagram for use of a sensor in a method for controlling flow in an arteriovenous graft.

FIG. 16 is a schematic view of a portion of an arteriovenous graft including a plurality of piezoelectric elements and a sensor spaced along a body portion of the arteriovenous graft.

FIG. 17 is a schematic view of the arteriovenous graft as shown in FIG. 6 showing needle insertion.

FIG. 18 is a perspective view of the arteriovenous graft as shown in FIG. 4 connected at each end to an artery.

### DESCRIPTION

The arteriovenous graft according to the present invention is for use in medical procedures requiring vascular access and, in particular, hemodialysis. The features described herein may be used with any conventional vascular access graft including such as, for example, the arteriovenous graft described by U.S. Pat. No. 9,585,998, the contents of which are hereby incorporated by reference herein in their entirety. A similar vascular access graft and application is shown and described in U.S. Pat. No. 7,833,186 and U.S. Pub. Application No. 2009/0209921, the contents of both of which are also incorporated by reference herein in their entirety. U.S. Pat. No. 8,764,698, the contents of which are hereby incorporated by reference herein in their entirety, describes various means for controlling blood flow through an arteriovenous graft. Accordingly, detailed explanations of the functioning of all of the components and use of the vascular access grafts are deemed unnecessary for understanding of the present description by one of ordinary skill in the art.

Certain terminology is used herein for convenience only and is not to be taken as a limiting. For example, words such as “upper,” “lower,” “left,” “right,” “horizontal,” “vertical,” “upward,” “downward,” “top” and “bottom” merely describe the configurations shown in the FIGs. Indeed, the components may be oriented in any direction and the terminology, therefore, should be understood as encompassing such variations unless specified otherwise. The words “interior” and “exterior” refer to directions toward and away from, respectively, the geometric center of the core and designated parts thereof. The terminology includes the words specifically mentioned above, derivatives thereof and words of similar import.

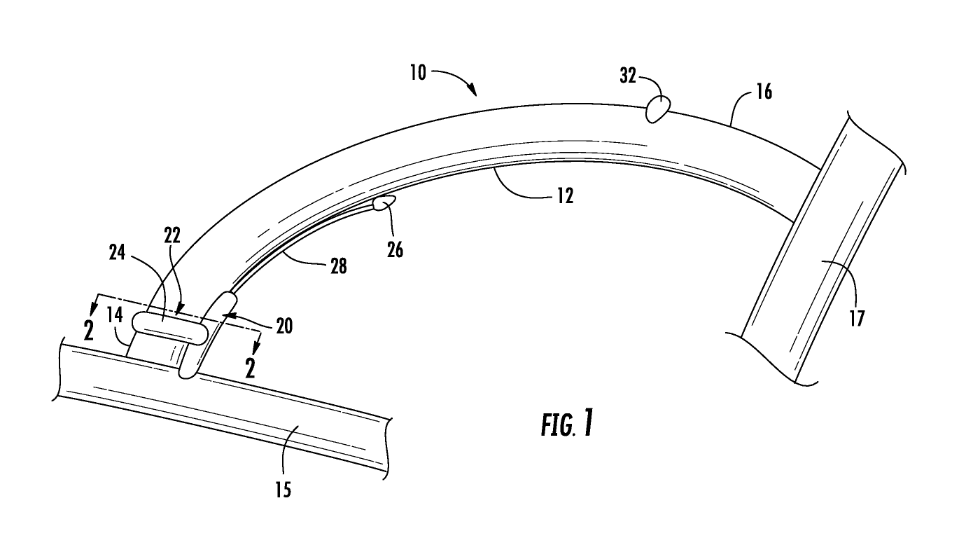

Referring now to FIG. 1, an embodiment of a subcutaneous arteriovenous graft for connecting an artery to a vein is shown and generally designated at 10. The arteriovenous graft 10 includes a tubular body portion 12 having an arterial end 14 and an opposite venous end 16. The arterial end 14 is configured to be connected to an artery 15 to form an arterial anastomosis, while the venous end 16 is configured to be connected to a vein 17 to form a venous anastomosis.

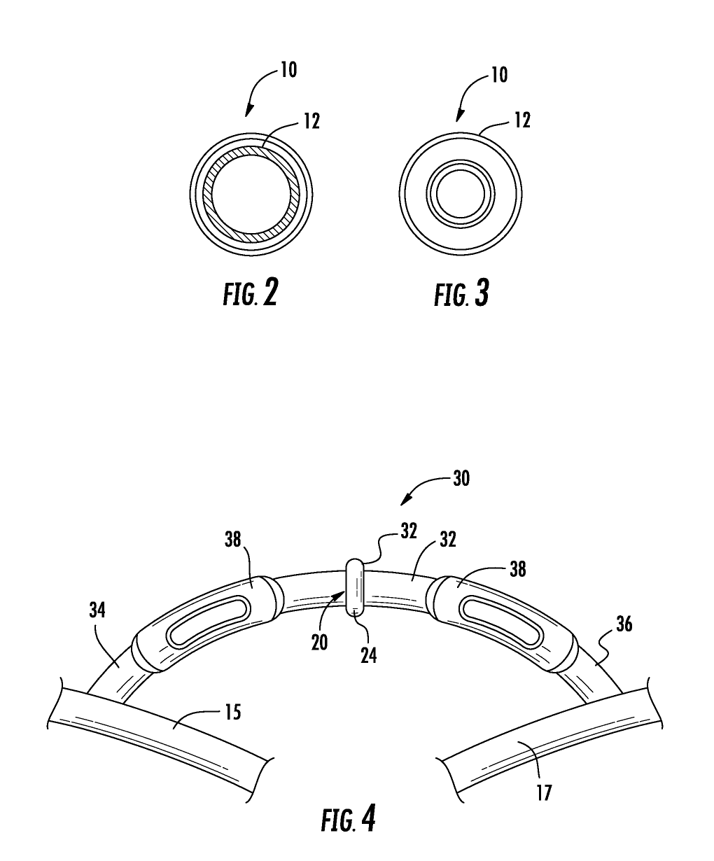

A valve device 20 is placed along the body portion 12 of the arteriovenous graft 10. The valve device 20 has an open position and a closed position. The valve device 20 functions in an open position for allowing blood flow through the arteriovenous graft 10 during normal hemodialysis. During hemodialysis, two hypodermic needles are inserted through the skin and into the arteriovenous graft 10. Blood is removed from the arteriovenous graft 10 using one needle, circulated through a dialysis machine and returned to the arteriovenous graft through the second needle. When hemodialysis has ended, the valve device 20 is moved from the open position (FIG. 2) toward the closed position (FIG. 3) in order to reduce blood flow through the arteriovenous graft 10. In this manner, arterial steal is being minimized as the valve device 20 constricts the body portion 12 of the arteriovenous graft 10. Blood flow through the body portion 12 continues without clotting, but at a reduced flow rate. Alternatively, the balloon 22 may be inflated such that blood flow through the arteriovenous graft 10 is prevented. In this position, the valve device 20 is fully closed and the inflated balloon 22 constricts the body 12 off the arteriovenous graft 10 for preventing arterial blood flow through the arteriovenous graft. The valve device 20 is configured for maintaining its position when exposed to systolic pressure. In both cases, the occurrence of arterial steal and graft thrombosis are minimized.

As shown in FIG. 1, the valve device 20 is positioned at the arterial end 14 of the arteriovenous graft 10 adjacent the intersection of the arteriovenous graft arterial end 14 and the artery 15. In one embodiment, the valve device 20 comprises an inflatable balloon assembly 22. The balloon assembly 22 is positioned so that when inflated the balloon assembly 22 restricts or eliminates blood flow through the body portion 12 of the arteriovenous graft 10. The balloon assembly 22 may have an annular shape that surrounds the body portion 12 of the arteriovenous graft 10. It is understood the balloon may have any shape sufficient to constrict the body portion 12 of the arteriovenous graft 10 when inflated. When the balloon 22 is inflated, the balloon 22 forces together opposite sides of the body portion 12. The balloon 22 may be surrounded by a rigid collar 24 that holds the balloon 22 in position and serves to assist the balloon in constricting the body portion 12 of the arteriovenous graft 10. The collar 24 may be formed from any biocompatible rigid material, for example a metal, such as titanium, or a plastic material.

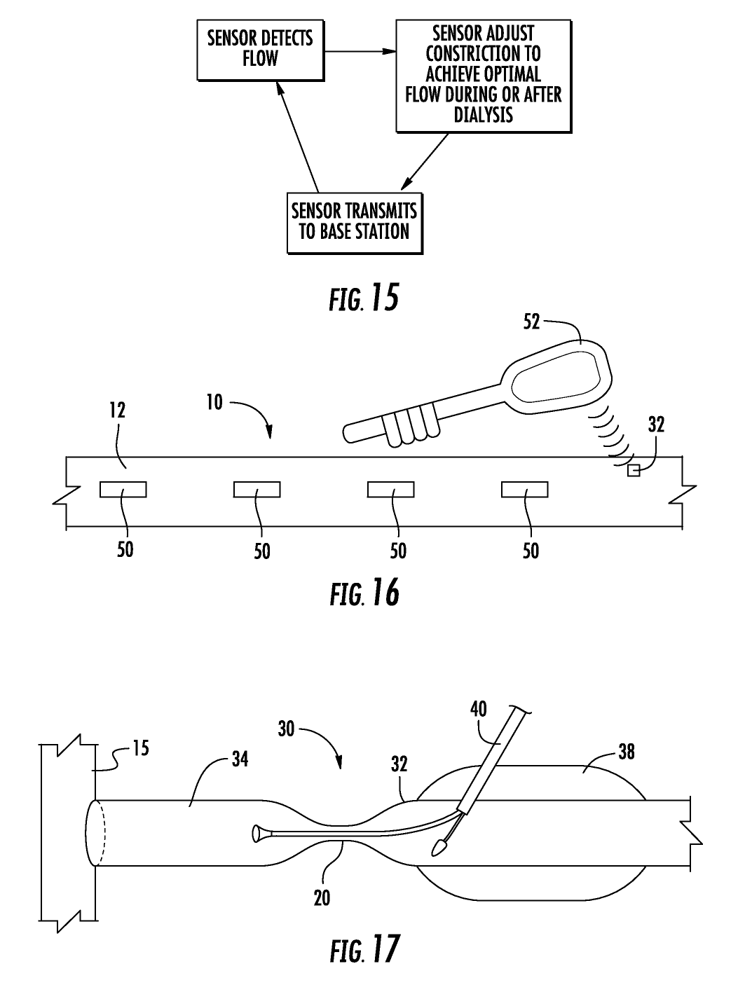

In order to inflate and deflate the balloon assembly 22, the valve device 20 may further comprise an injection port 26 in fluid communication with the inflatable balloon 22 via tubing 28. The injection port 26 may be subcutaneously implanted with the arteriovenous graft 10. The injection port 26 defines a target area configured to receive a hypodermic needle (not shown) for injecting fluid into, or withdrawing fluid from, the balloon 22. Fluid to inflate the balloon 22 travels from the injection port 26 through the tubing 28 and into the balloon 22. The fluid used to inflate the balloon 22 may be, for example, a gas or a liquid. In one embodiment, a saline solution may be injected into the injection port 26 for inflating the balloon 22 after hemodialysis has ended. Although the injection port 26 is shown in one embodiment as being subcutaneously implanted with the arteriovenous graft 10, it is understood that the injection port 26 and at least a portion of the associated tubing 28 may extend externally of the skin surface for facilitating fluid injection or removal from the balloon 22 (FIG. 14).

FIG. 4 is a perspective view of another embodiment of an arteriovenous graft which is generally designated at 30. The vascular access graft 30 includes a tubular body portion 32 of biocompatible material for conducting fluid such as blood. As is conventional, the tubular portion 42 is anastomosed at a first arterial end 34 to an artery 15 and anastomosed to a vein 17 at a second venous end 36. A pair of spaced cannulation chambers 38 are disposed intermediate along the length of the body portion 32 in fluid communication with the arterial side and the venous side of the arteriovenous graft 30.

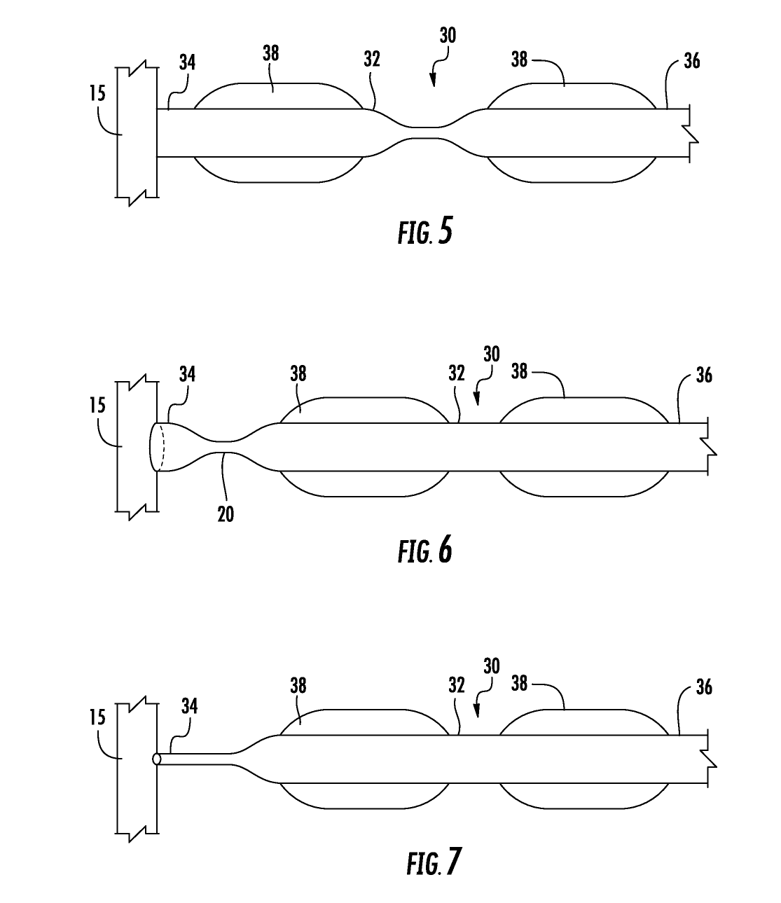

A valve device 20 as described hereinabove is positioned between the cannulation chambers 38. As schematically shown in FIG. 5, the valve device 20 constricts blood flow through the body portion 12 of the arteriovenous graft 10 when hemodialysis is not occurring. Because a graft with cannulation chambers as described by U.S. Pat. No. 9,585,998 has specific zones for cannulation, the valve device 20 can now be placed anywhere along the body portion 32 the graft 30 without fear of accidental cannulation of the valve device 20. Moreover, while placing a valve or constriction away from an arterial anastomosis could increase the pressure observed in the arterial side of the graft 30, because the cannulation chamber 38 contains self-sealing material and a backplate to avoid backwall punctures, a high pressure area could be cannulated, in contrast to standard clinical practice where it would not be advised to cannulate a high pressure area due to extended bleeding times and increased probabilities of hematoma formation.

FIG. 6 a schematic view of the arteriovenous graft 30 as shown in FIG. 4 with a valve device 10 at an arterial end 34 of the arteriovenous graft between the artery 15 and the cannulation chambers 32 with the valve shown in a closed position. With the valve device 20 disposed at the arterial end 34, the valve device 20 in this embodiment constricts blood flow through the cannulation chambers 38 when hemodialysis is not occurring. FIG. 7 shows a similar arrangement wherein at least part of the body portion 32 of the arteriovenous graft 30 has a narrower diameter beginning at the artery 15.

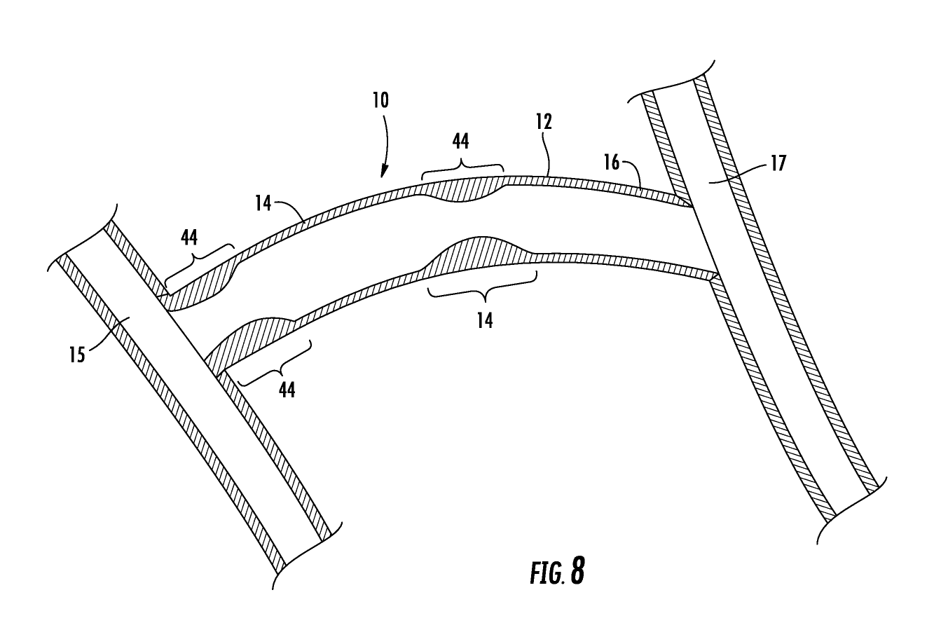

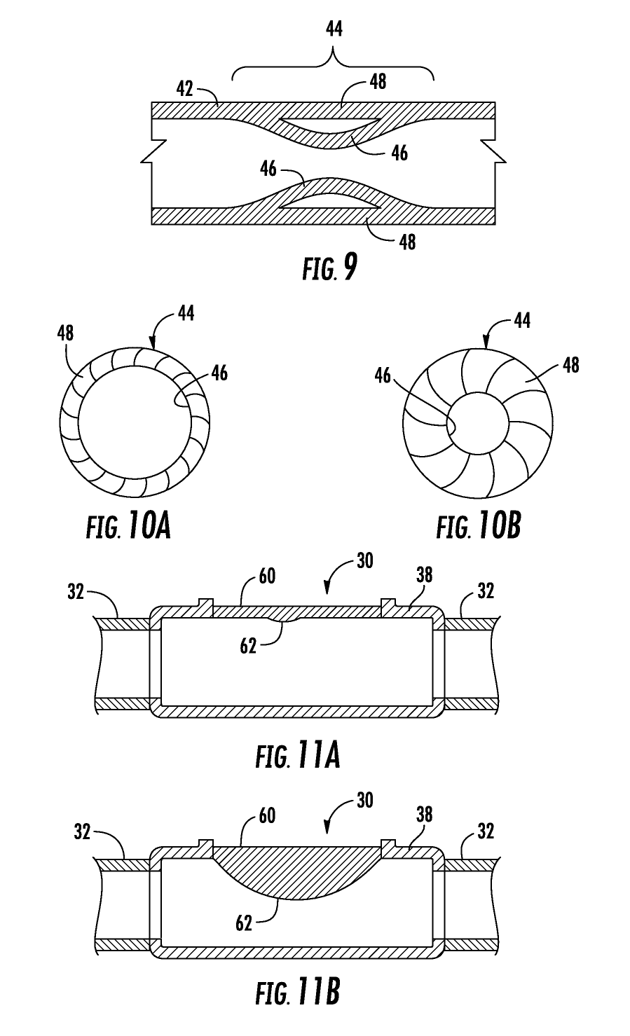

Referring to FIGS. 8 and 9, an embodiment of the arteriovenous graft 10 is shown wherein the inflatable balloon 22 is integral with the body portion 12 of the graft. In this embodiment, the walls 13 of the body portion 12 may include a multi-layered segment located, for example, at the arterial end 14 as shown in FIG. 8. The multi-layered segment 44 may comprise an inner luminal 46 layer and an outer layer 48. The balloon 22 may be formed between the inner layer 46 and the outer layer 48. When a fluid is injected between the inner layer 46 and the outer layer 48, the inner layer 46 will expand and constrict the lumen defined by the body portion 12 of the arteriovenous graft 10.

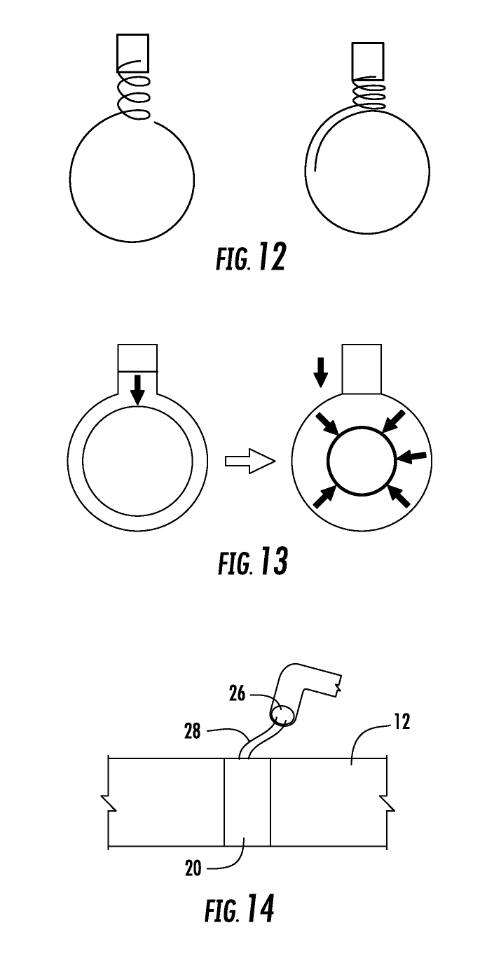

FIGS. 11A and 11B show an up-close longitudinal cross-section view of the arteriovenous graft as shown in FIG. 4, including an embodiment of a variable diameter cannulation chamber 38. The cannulation chamber 38 comprises a valve device 60 having a variable diameter layer 62 lining the cannulation chamber 38. The valve device 60 is shown in an open position (FIG. 11A) and a closed position (FIG. 11B).

As seen in FIG. 1, the arteriovenous graft 10 may comprise electronic pressure and flow sensors 32 in the arterial end 14 or venous end 16 of the body portion 12. The sensors 32 are electronically coupled to a monitor the arterial and venous environments and change the degree of stenosis or narrowing of the arteriovenous graft 10 to optimize flow. The stenosis or narrowing within the body portion 12 creates resistance for the purpose of decreasing the flow rate and pressure at the venous end 16 of the arteriovenous graft 12. Monitoring flow in this manner using sensors 32 allows control of the degree of stenosis by adjusting, for example, the balloon 22 inflation level to optimize flow of fluid through the arteriovenous graft 10. Monitoring of the sensors may include transmission of signals to a base station for centralized monitoring (FIG. 15).

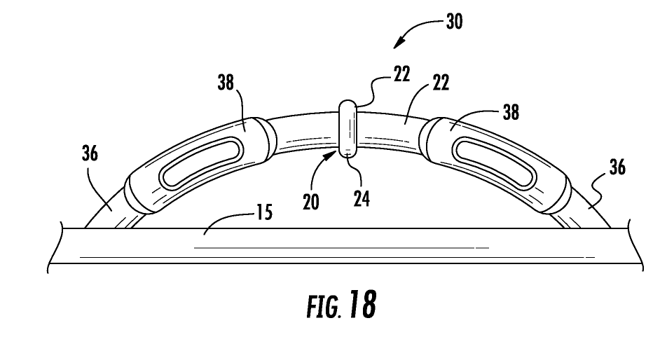

FIG. 16 is an elevation view of the arteriovenous graft 10 as shown in FIG. 1. In this arrangement, the arteriovenous graft 10 includes a plurality of piezoelectric elements 50 longitudinally spaced along the body portion 12. A wand 52 may be used to activate the piezoelectric elements 50 for vibrating the body 12 and encouraging blood flow and minimizing clotting.

FIG. 17 shows an embodiment of a device to bypass a valve device or other constriction in order to access an area of increased blood flow or to gain separation from the point of needle 40 entry. During hemodialysis, a certain separation is required between the inflow and outflow needle to prevent recirculation. Recirculation occurs when dialyzed blood returning through the venous needle re-enters the extracorporeal circuit through the arterial needle rather than returning to systemic circulation. In this embodiment, a needle-like device 40 gains access to the vascular access circuit. The needle-like device 40 comprises one or more openings to allow a second device to extend outwardly and travel upstream or downstream in the vascular access circuit. This embodiment allows a user to bypass a constriction or valve in a vascular access circuit to access an area of greater blood flow. This embodiment could also be used to place two very close vascular access points, wherein the second device would pass through and travel upstream or downstream in the vascular access circuit to gain appropriate separation from the first device. The second device is partially or wholly made of flexible material to facilitate passage through the needle-like device 40. Because the second device is not designed to gain entry into the vascular access circuit, the second device does not need to be sharp, and can also have unique tip geometry to increase the capability of the second device to pull blood through the second device.

FIG. 18 is a perspective view of the arteriovenous graft 30 as shown in FIG. 4. In this arrangement, the body portion 32 of the arteriovenous graft 30 is anastomosed at a first arterial end 34 to an artery 15 and anastomosed at the second end 36 to the same artery 15. A valve device 20 may be positioned between the cannulation chambers 32 for constricting blood flow through the body portion 32 of the arteriovenous graft 30 when hemodialysis is not occurring. Because a cannulation chamber, such as the cannulation chambers disclosed in the Gage patent comprise self-sealing material and a backplate to avoid backwall punctures, a high pressure area can be cannulated, in contrast to standard clinical practice where it would not be advised to cannulate a high pressure area due to extended bleeding times and increased probabilities of hematoma formation.

The arteriovenous graft systems as described herein have many advantages, including preventing or minimizing arterial steal and graft thrombosis. In particular, the arteriovenous graft systems are designed to prevent or minimize blood flow through the graft when hemodialysis is not occurring. Reducing or stopping blood flow through the arteriovenous graft when hemodialysis is not occurring may also prevent the graft from leaking when the hypodermic needles used to carry out hemodialysis are removed from the graft.

Although the present invention has been shown and described in considerable detail with respect to only a few exemplary embodiments thereof, it should be understood by those skilled in the art that I do not intend to limit the invention to the embodiments since various modifications, omissions and additions may be made to the disclosed embodiments without materially departing from the novel teachings and advantages of the invention, particularly in light of the foregoing teachings. For example, the present invention is suitable for use in a number of vascular access devices and applications. Accordingly, we intend to cover all such modifications, omission, additions and equivalents as may be included within the spirit and scope of the invention as defined by the following claims. In the claims, means-plus-function clauses are intended to sticker the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

We claim:

1. An arteriovenous dialysis access graft configured to be implanted in a body of a subject, the arteriovenous dialysis graft comprising:
a flexible conduit defining a longitudinal flow passageway, the conduit having a first end portion and a second end portion the first end portion is configured to connect to an artery of the subject and the second end portion configured to connect to a vein of the subject such that blood flows through the longitudinal flow passageway of the conduit from the first end portion to the second end portion;
a first cannulation chamber and a spaced second cannulation chamber with the conduit extending through each of the first chamber and the second chamber, the first chamber positioned between the first end portion and the second chamber, and the second chamber positioned between the second end portion and the first chamber; and
a valve device is positioned between the first cannulation chamber and the second cannulation chamber for controlling fluid flow.

2. The arteriovenous dialysis graft as recited in claim 1, wherein the valve device comprises an inner sleeve positioned within an outer sleeve, and an actuator in flow communication with the fluid passageway defined through the outer sleeve of the valve device, the actuator being configured to open and close the valve device when fluid is directed through the fluid passageway and against the wall portion such that the wall portion inflates so as to form a balloon that restricts the flow of blood through the arteriovenous graft.

3. The arteriovenous dialysis graft as recited in claim 2, wherein the actuator is positioned outside of the arteriovenous graft

4. The arteriovenous dialysis graft as recited in claim 2, wherein the actuator comprises a fluid injection port that is in fluid communication with the valve device.

5. The arteriovenous dialysis graft as recited in claim 2, wherein the outer sleeve of the valve device is more rigid than the inner sleeve and wherein the outer sleeve maintains its shape when the respective balloon is inflated.

6. The arteriovenous dialysis graft as recited in claim 2, wherein the actuator comprises a piston that pumps the fluid to the discrete area of each valve device.

7. The arteriovenous dialysis graft as recited in claim 2, wherein the actuator comprises a fluid delivery device that delivers the fluid to the valve device for opening and closing the valve device.

8. The arteriovenous dialysis graft as recited in claim 7, wherein the fluid comprises a liquid.

9. The arteriovenous dialysis graft as recited in claim 7, wherein the fluid comprises a gas.

10. The arteriovenous dialysis graft as recited in claim 1, wherein the fluid comprises a liquid.

11. The arteriovenous dialysis graft as recited in claim 1, wherein each chamber comprises
an elongated chamber body surrounding the conduit, the chamber body comprising
an annular inner layer including self-sealing material surrounding the conduit, and
an outer layer around the inner layer and defining a cannulation port that exposes the self-sealing material; and
an elongated shell embedded in the chamber body between the inner layer of the chamber body and the outer layer of the chamber body and extending generally parallel to the longitudinal flow passageway of the conduit, the shell including a posterior wall and a pair of sidewalls defining an open anterior portion facing the cannulation port of the chamber body, wherein each shell is formed of a substantially rigid material such that, when a dialysis needle is inserted through the cannulation port and the self-sealing material, the needle is inhibited or prevented from extending through the posterior or the side walls of the shell.

12. An arteriovenous dialysis access graft configured to be implanted in a body of a subject, the arteriovenous dialysis graft comprising:
a flexible conduit defining a longitudinal flow passageway, the conduit having a first end portion and a second end portion the first end portion is configured to connect to an artery of the subject and the second end portion configured to connect to a vein of the subject such that blood flows through the longitudinal flow passageway of the conduit from the first end portion to the second end portion;
a cannulation chamber with the conduit extending through the chamber, the cannulation chamber positioned between the first end portion and the second end portion; and
a valve device is positioned between the cannulation chamber and the second end portion for controlling fluid flow.

13. The arteriovenous dialysis graft as recited in claim 12, wherein the valve device comprises an inner sleeve positioned within an outer sleeve, and an actuator in flow communication with the fluid passageway defined through the outer sleeve of the valve device, the actuator being configured to open and close the valve device when fluid is directed through the fluid passageway and against the wall portion such that the wall portion inflates so as to form a balloon that restricts the flow of blood through the arteriovenous graft.

14. The arteriovenous dialysis graft as recited in claim 13, wherein the actuator is positioned outside of the arteriovenous graft

15. The arteriovenous dialysis graft as recited in claim 13, wherein the actuator comprises a fluid injection port that is in fluid communication with the valve device.

16. The arteriovenous dialysis graft as recited in claim 13, wherein the outer sleeve of the valve device is more rigid than the inner sleeve and wherein the outer sleeve maintains its shape when the respective balloon is inflated.

17. The arteriovenous dialysis graft as recited in claim 13, wherein the actuator comprises a piston that pumps the fluid to the discrete area of each valve device.

18. The arteriovenous dialysis graft as recited in claim 13, wherein the actuator comprises a fluid delivery device that delivers the fluid to the valve device for opening and closing the valve device.

19. The arteriovenous dialysis graft as recited in claim 7, wherein the fluid comprises a liquid or a gas.

20. The arteriovenous dialysis graft as recited in claim 12, wherein the cannulation chamber comprises
an elongated chamber body surrounding the conduit, the chamber body comprising
an annular inner layer including self-sealing material surrounding the conduit, and
an outer layer around the inner layer and defining a cannulation port that exposes the self-sealing material; and
an elongated shell embedded in the chamber body between the inner layer of the chamber body and the outer layer of the chamber body and extending generally parallel to the longitudinal flow passageway of the conduit, the shell including a posterior wall and a pair of sidewalls defining an open anterior portion facing the cannulation port of the chamber body, wherein each shell is formed of a substantially rigid material such that, when a dialysis needle is inserted through the cannulation port and the self-sealing material, the needle is inhibited or prevented from extending through the posterior or the side walls of the shell.
